(19) United States
(12) Patent Application Publication
(10) Pub. No.: US 2012/0283679 A1
(43) Pub. Date: Nov. 8, 2012

(54) Belly binder with a reinforced opening

(76) Inventors: Mary L. Berish, Belle Vernon, PA (US); Brett R. Berish, Belle Vernon, PA (US)

(21) Appl. No.: 13/068,010

(22) Filed: May 2, 2011

(51) Int. Cl. A61F 5/449

(52) U.S. Cl. 604/345

(57) ABSTRACT

A belly binder for providing support to an ostomy apparatus is disclosed. The belly binder comprises a flexible band, preferably made of a heavy duty elastic material. The band is wrapped around the abdomen of a wearer for support. Fasteners are attached to the ends of the flexible band to hold the band in position. The flexible band comprises, on its surface, an opening that is adapted to receive an ostomy bag. Further, a support member is attached to the flexible band. The support member is such that it forms a pocket/pouch like structure to hold and support the ostomy bag.

In one embodiment, the binder can also be in the form of a girdle as a less bulky alternative for individuals concerned about modesty.

### FIELD OF THE INVENTION

The present invention generally relates to a belly binder and more particularly, to an improved belly binder for securely supporting an ostomy bag or pouch. The binder is designed to support the ostomy collection bag in a secure position on the patient's abdomen. This is achieved without interfering with the functions of the ostomy bag.

### BACKGROUND OF THE INVENTION

An ostomy is a surgically created opening on an abdominal wall through which waste material passes out of the body. An ostomy bag (a pouching system) is a medical device prosthetic that provides a means for the collection of this waste material.

People who have an ostomy face numerous difficulties due to their medical condition. The various difficulties are usually in handling drainages that leave the body through a stoma provided surgically on the skin, typically in the abdominal region. As the bag fills up with the bodily drainages, its weight increases. This weight exerts a downward pull on the skin surrounding the stoma that causes skin irritation. Also, due to the pull, the ostomy bag can become detached from the body.

Further, the pouching systems are usually attached mechanically or with an adhesive in an airtight seal. Any type of relative movement between the surrounding skin and the adhesive material can create an opening through which the bodily drainages can leak. Such leakages are a source of embarrassment, smell and damage to clothing of the person. They also pose a health hazard to third parties coming in contact with the bodily discharges and an infection hazard for the patient.

There exist binders in the prior art that hold the ostomy bag close to the stomach. Such binders are wrapped around the wounded region, with the ostomy bag intact inside. However, as the bag fills up, it causes pressure to build up and hence the ostomy bag can rupture. Also, as the ostomy bag is not accessible without removing the binder. Such binders are not useful in situations that require frequent inspections and ostomy bag changes.

According to U.S. Pat. No. 5,626,570 (Kerry R. Gallo), there is provided a binder with an opening for the ostomy bag, through which the bag can be replaced. However, the bag is kept horizontally and covered within a flap. This can lead to accumulation of excrement close to the stoma resulting in safety issues and leakages. The excrement needs to be away from the stoma which is best accomplished by keeping the ostomy bag vertical and facilitating the excrement discharge flow with gravity.

Hence, there exists a need for a system that addresses the aforementioned problems and provides a good support to the ostomy bag, with a good fit to different wearers.

### SUMMARY

This need is met with a novel belly binder to support an ostomy bag. The belly binder comprises a flexible band, a plurality of fasteners, a reinforced opening and a support pocket. The fasteners are attached at the ends of the flexible band to hold the band in position and to provide a custom fit. The opening is provided in the band and reinforced, so as to support the stoma area. In a preferred embodiment, the pocket is provided below the reinforced opening so that the ostomy bag can be placed inside the pocket. This gives support to the bag and avoids rupturing of the bag.

In another embodiment, a girdle to support an ostomy bag is disclosed. The girdle comprises a main body, a plurality of fasteners, a reinforced opening and a support pocket. The main body is such that it confines the hip and abdomen region of a wearer. The fasteners are located along the vertical length of the girdle, to hold the girdle in position. The opening is provided in the flexible band to receive the ostomy bag. In a preferred embodiment, the pocket is provided below the reinforced opening so that the ostomy bag can be placed inside the pocket, to give the bag support and to avoid rupturing of the bag.

It is an object of the present invention to provide a belly binder that provides support to an ostomy bag, without obstructing or rupturing the ostomy bag.

It is another object of the present invention to provide a belly binder that provides a customized fit to a user.

It is yet another object of the present invention to provide a girdle that acts as a belly binder. This girdle provides all the advantages of the belly binder while working as a less bulky alternative for individuals concerned about modesty.

Whereas there may be many embodiments of the present invention, each embodiment may meet one or more of the foregoing recited objects in any combination. It is not intended that each embodiment will necessarily meet each objective. Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification.

### BRIEF DESCRIPTION OF THE DRAWINGS

Embodiments of the invention hereinafter described in conjunction with the appended drawings, which are provided to illustrate and not to limit the present invention, wherein like designations denote like elements, and in which:

FIG. 1 illustrates a belly binder that is laid out fully flat.

FIG. 2 shows a magnified view of the reinforced section of the belly binder.

FIG. 3 illustrates a close view of the hook and eye arrangement of the belly binder.

FIG. 4 illustrates a belly binder worn by a person.

FIG. 5 shows a belly binder in use, with the ostomy bag attached.

FIG. 6 shows another embodiment of a belly binder, as being used by a wearer.

FIG. 7 shows a cross-sectional view of the belly binder illustrating a reinforced opening, a support pocket and an ostomy bag.

FIG. 8 shows an embodiment of the belly binder in the form of a girdle.

FIG. 9 illustrates another embodiment of the girdle.

FIG. 10 illustrates yet another embodiment of the girdle.

FIG. 11 illustrates still another embodiment of the girdle.

The drawings are not to scale, in fact, some aspects have been emphasized for a better illustration and understanding of the written description.

### PARTICULAR ADVANTAGES OF THE INVENTION

The support member 106 holds the ostomy bag in position when the bag fills up from the bodily wastes. Further, the support member 106 protects the stoma from the gravitational pull of the ostomy bag. This reduces the risk of bag rupture and/or leakage. The belly binder comprises a flexible band, preferably made of a heavy duty elastic material. The band is wrapped around the abdomen of a wearer for support. Fasteners are attached to the ends of the flexible band to hold the band in position. The flexible band comprises, on its surface, an opening that is adapted to receive an ostomy bag. Further, a support member is attached to the flexible band. The support member is such that it forms a pocket/pouch like structure to hold and support the ostomy bag. In one embodiment, the binder can also be in the form of a girdle as a less bulky alternative for individuals concerned about modesty.

### DETAILED DESCRIPTION OF A PREFERRED EMBODIMENT

In the detailed description of the invention, numerous specific details are described to provide a thorough understanding of the various embodiments of the invention. However, one skilled in the relevant art will recognize that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the invention.

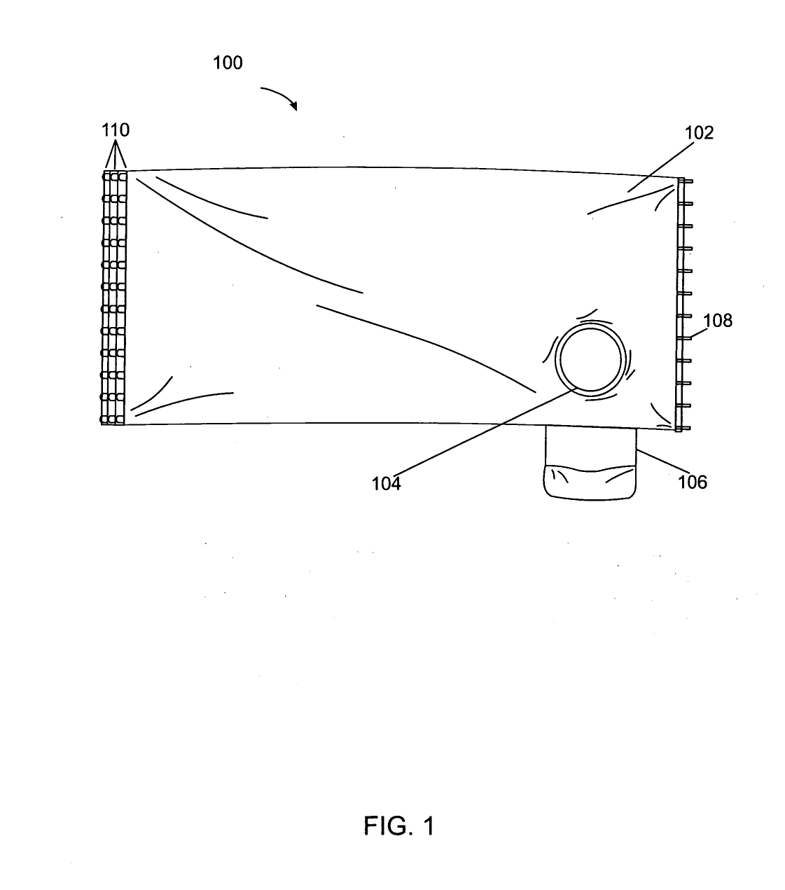

FIG. 1 illustrates a belly binder 100 that is laid out fully flat. A belly binder 100 is an abdominal support belt that can be worn after an abdominal surgery like ileostomy, colostomy or urostomy to aid recovery. In such surgeries, an artificial opening, also known as a stoma, is surgically created on the abdomen wall to remove the bodily wastes out of the body. This surgical procedure is invoked usually as a result of and solution to disease in the gastrointestinal tract. The procedure involves bisecting this tube, usually between the later stage of the small intestine (ileum) and the large intestine or colon, and exiting it from the body in the abdominal region. The point of exiting is what is known as the stoma.

The healing of such surgeries may be achieved by covering the stoma by a belt and providing support to the surrounding skin. FIG. 1 shows such an exemplary belt—a belly binder 100. The belly binder 100 comprises a flexible band 102, an opening 104, a support member 106, and a plurality of fasteners 108 and 110. The flexible band 102 forms the main portion of the belly binder 100. The flexible band 102 is wrapped around the waist or the mid-section following an abdominal surgery to aid in the healing process of the muscles of the abdominal wall. The flexible band 102 is preferably made of an elastic material, so as to provide good support and fit to a user. Examples of the material used to form the flexible band 102 include, but are not limited to, polyester, spandex, lastol, LYCRA®, ELASPAN (Invista), CREORA (Hyosung), ROICA (Asahi Kasei), and DORLASTAN (Asahi Kasei), LINEL (Fillattice), and ESPA (Toyobo), and the like. In some aspects, the material is treated by methods known in the art to provide waterproofing, antimicrobial, deodorant and other desirable characteristics. Preferably, the material is washable. In some aspects, the material is disposable or single-use type medical grade material.

The flexible band 102 comprises the opening 104. The opening 104 is adapted to receive a medical prosthetic device. In an embodiment, the medical prosthetic device is an ostomy bag. The opening 104 can be of any shape. The shape of the opening 104 may be circular, square, rectangular, elliptical etc. FIG. 1 shows an opening 104 that is circular in shape. The opening 104 is reinforced at its circumference to provide support to the stoma and to maintain integrity of the flexible band 102. In an embodiment, the opening 104 is reinforced by using a pliable plastic that is placed between two layers of elastic binding material. Further, the opening 104 maybe reinforced from both sides of the flexible band 102, inside as well as outside. The opening 104 is of a size such that it can easily receive an ostomy bag, without interfering or rupturing the ostomy bag. In an embodiment, the opening 104 is such that the diameter of the opening 104 is greater than the diameter of the ostomy bag (attached to the body). The provision of the opening 104 on the surface of the flexible band 102 makes the ostomy bag accessible without the removal of the belly binder 100.

The flexible band 102 further comprises the support member 106. The support member 106 is such that it forms a pocket that hangs vertically down from the flexible band 102. The support member 106 holds the ostomy bag in position when the bag fills up from the bodily wastes. Further, the support member 106 protects the stoma from the gravitational pull of the ostomy bag. This reduces the risk of bag rupture and/or leakage. The support member 106 is attached to the lower portion of the flexible band 102 and can be positioned anywhere on the flexible band 102. In a preferred embodiment, the support member 106 is attached close to the reinforced opening 104. As shown in FIG. 1, the support member 106 is attached vertically below the opening 104. In another embodiment, the support member 106 may also be attached directly to the opening 104.

Referring to FIG. 1, the belly binder 100 further comprises a plurality of fasteners 108, and 110. The fasteners 108, 110 are placed at either ends respectively of the flexible band 102. The fasteners 108 and 110 securely hold the belly binder 100 together and provide a good fit to the wearer. Further, it prevents a relative movement between the skin of a wearer and the belly binder 100. A series of fasteners are arranged along the width of the flexible band 102. Examples of the fastener arrangement 108, 110 include, but are not limited to, hook and eye arrangement, zipper arrangement, lace-thread arrangement, VELCRO® and a button-hole arrangement. Although only a single row of hooks is depicted in the exemplary drawings, it is to be appreciated that any number or arrangement of fasteners may be suitable adapted to provide the closing function. Multiple rows of fasteners may increase the compressive forces of the garment or distribute the pulling forces over a greater area to enhance the longevity of the fasteners and decrease wear and tear.

FIG. 1 represents a hook and eye arrangement. In an embodiment of the present invention, there is provided three series of eyeholes at one end of the flexible band 102. The plurality of fasteners 110 represent three-series eyeholes placed at three different locations along the width of the flexible band 102. Whereas the plurality of fasteners 108 represent a single series of hooks placed at the other end of the flexible band 102. This is done in order to provide three different fitting settings to the user for a close and snug fit of the belly binder 100.

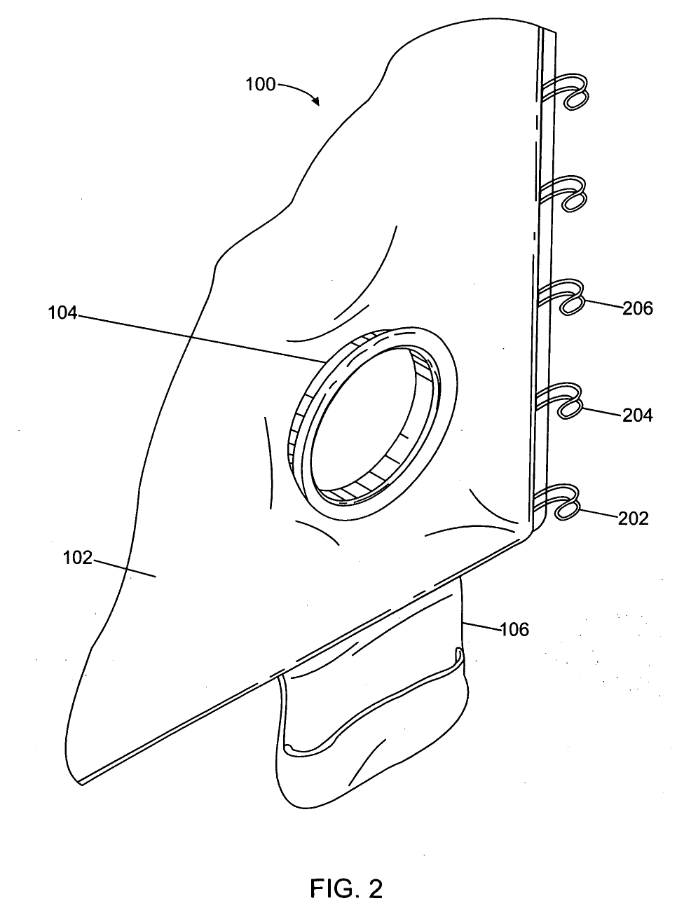

FIG. 2 shows a magnified view of the reinforced section of the belly binder 100. The magnified section shows a portion of the flexible band 102, the opening 104, the support member 106, and the plurality of fasteners 108. The plurality of fasteners 108 comprises a hook 202, a hook 204, a hook 206, and so on As shown in. FIG. 2, the reinforcement of the opening 104 spans across the two sides of the flexible band 102. The reinforcement is achieved by using a pliable plastic that is placed between the layers of material of the flexible band 102. The reinforcement of the opening 104 provides a better abdominal seal that helps in a quicker healing process. Further, it decreases any chance of leakage or rupturing of the ostomy bag. In an embodiment, the support member 106 is placed directly below the opening 104. The hooks 202, 204, 206 are heavy duty hooks used for binding and are placed along one end of the flexible band 102. In an embodiment, hooks 202, 204, and 206 are metal hook connectors.

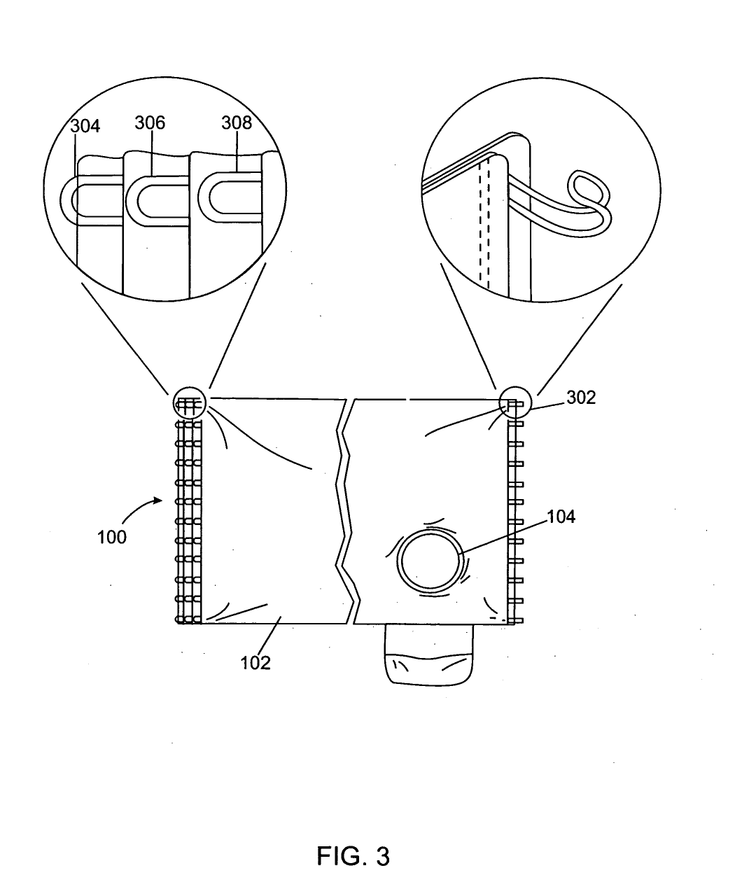

FIG. 3 illustrates a close view of the hook and eye arrangement of the belly binder 100. The arrangement shown comprises the flexible band 102, the opening 104, the support member 106, a hook 302, and the plurality of fasteners 110. The plurality of fasteners 110 further comprises an eye fixture 304, an eye fixture 306, and an eye fixture 308. The eye fixtures 304, 306, and 308 are located at three different positions along the length of the flexible band 102. This is done to provide 3-different fitting settings to a user. For example, the hook 302 can be engaged in the eye fixture 304 to achieve a fit. Further, to get a tighter fit (depending on the waist of the user) the hook 302 can also be engaged in the eye fixture 306, or the eye fixture 308. In an embodiment, the eye fixtures 304, 306, and 308 are U-clasps.

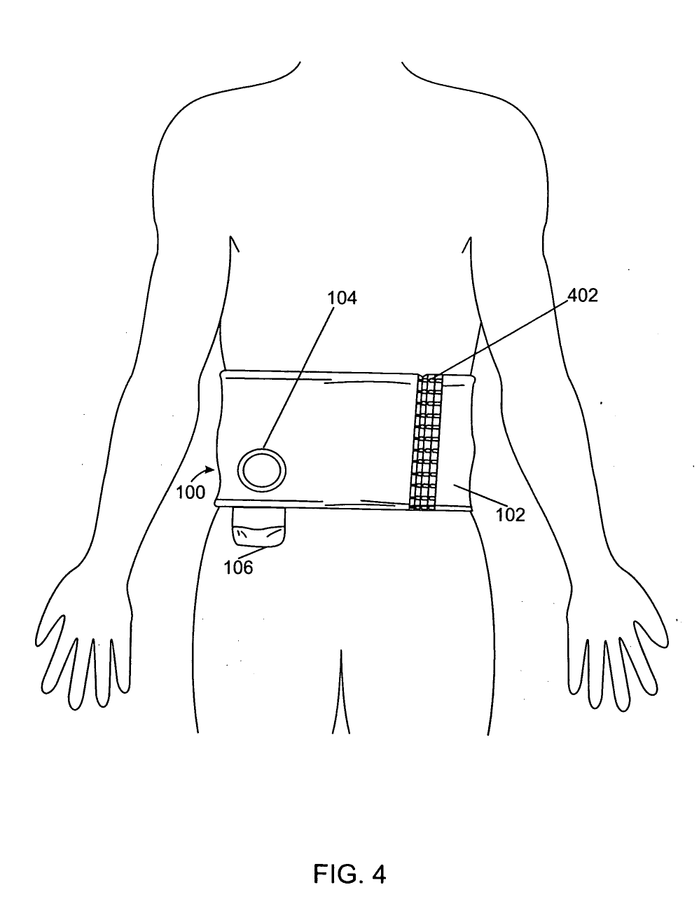

FIG. 4 illustrates the belly binder 100 as worn by a person. In the shown embodiment, the ostomy bag is placed on the right side of the body of the user. The belly binder 100 can be worn by placing the flexible band 102 on the back and bringing the two ends, of the flexible band 102 forward (as the case fora normal belt). The flexible band 102 is wrapped around the abdominal/mid section area in such a way that the opening 104 is placed over the area where the ostomy bag is placed. Before fastening the two ends, the ostomy bag can be pulled out through the reinforced opening 104. The flexible band 102 can then be secured with the fastening arrangement 402. In an embodiment the fastening arrangement 402 are a plurality of hook and eye fixtures. The arrangement of the fasteners 402 is same as have been previously explained in FIGS. 1 and 3. After pulling out the ostomy bag through the reinforced opening 104, the end of ostomy bag can be placed in the support member 106. In a preferred embodiment, the ostomy bag stays vertical in the preferred embodiment, with the support member 106 positioned vertically below the opening 104. In an alternate embodiment, the support member 106 can be positioned anywhere along the length of the flexible band 102.

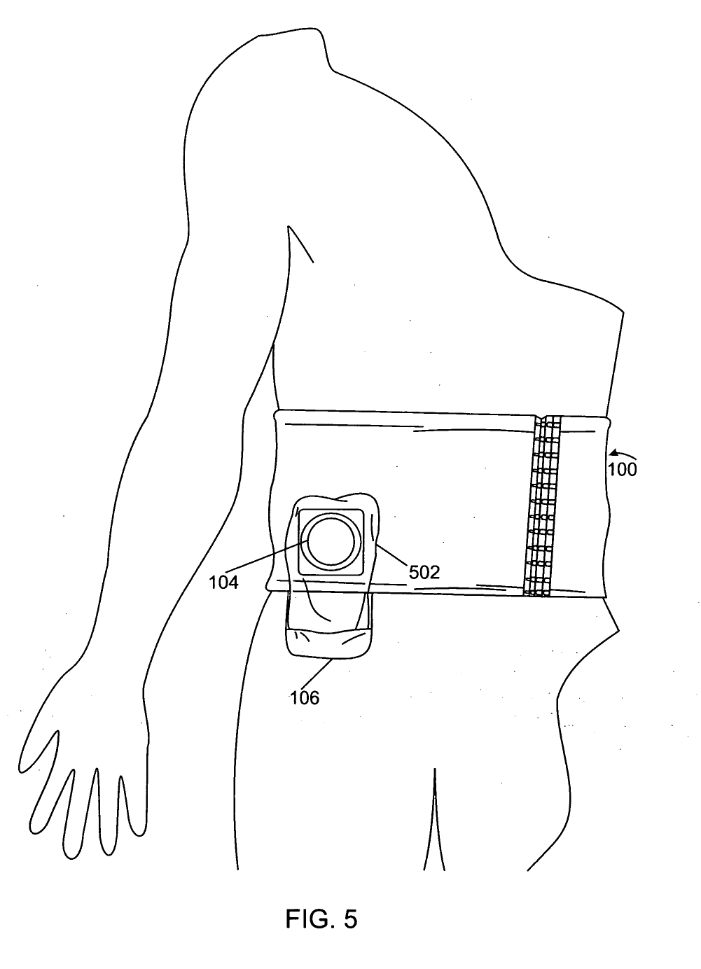

FIG. 5 shows a belly binder in use, with an ostomy bag attached. As shown, an ostomy bag 502 is supported by the belly binder 100. The ostomy bag 502 is a medical device prosthetic that helps in the collection of wastes from a surgically diverted biological system. The ostomy bag 502 is mostly used after surgeries like colostomy, ileostomy, and urostomy. FIG. 5 shows the belly binder 100 with the ostomy bag 502 received in the opening 104. After the ostomy bag 502 is pulled through the opening 104, the bottom of the ostomy bag 502 is placed on the support member 106. In a preferred embodiment, the support member 106 is a pocket/pouch like structure. The reinforced opening 104 provides support to the abdominal area without obstructing or rupturing the ostomy bag 502, since the ostomy bag 502 is outside the belly binder 100. Further, the support member 106 prevents the ostomy bag 502 to fall down due to the gravitational pull of the bodily wastes.

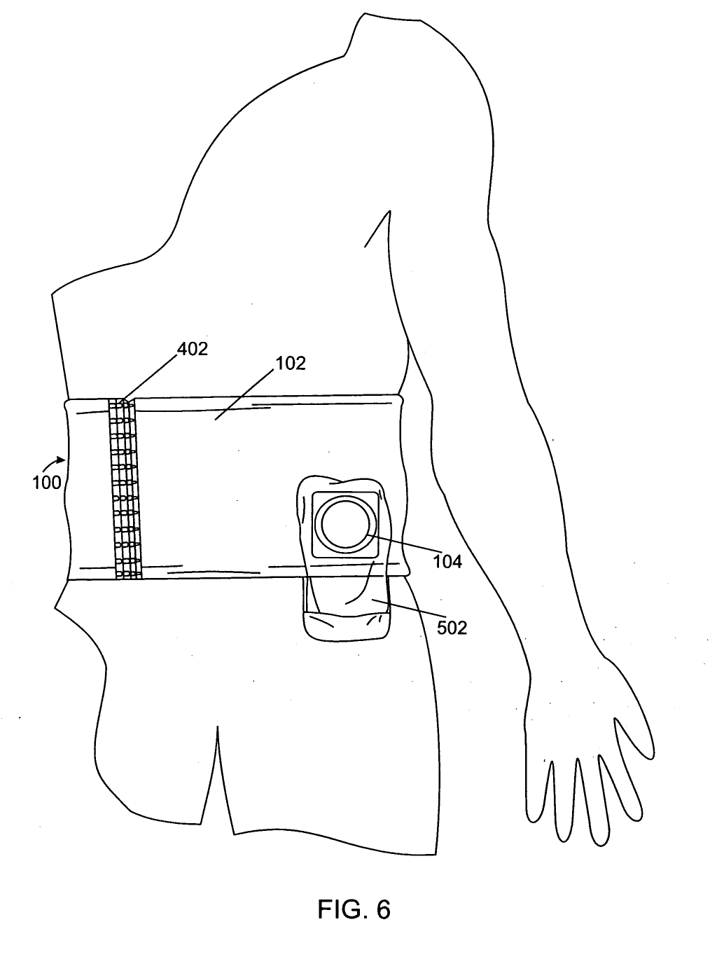

FIG. 6 shows another embodiment of the belly binder 100, as being used by a wearer. In the shown example, the ostomy bag 502 is located on the left side of the body of the wearer. It should be observed that the belly binder 100 can receive the ostomy bag 502 conveniently, irrespective of the position of the ostomy bag 502. In an embodiment, this can be achieved by placing the opening 104 close to the edge of the flexible band 102. In this way, after adapting the opening 104 to the ostomy bag 502, the fastener arrangement 402, still remains accessible to the wearer to engage the fastener arrangement.

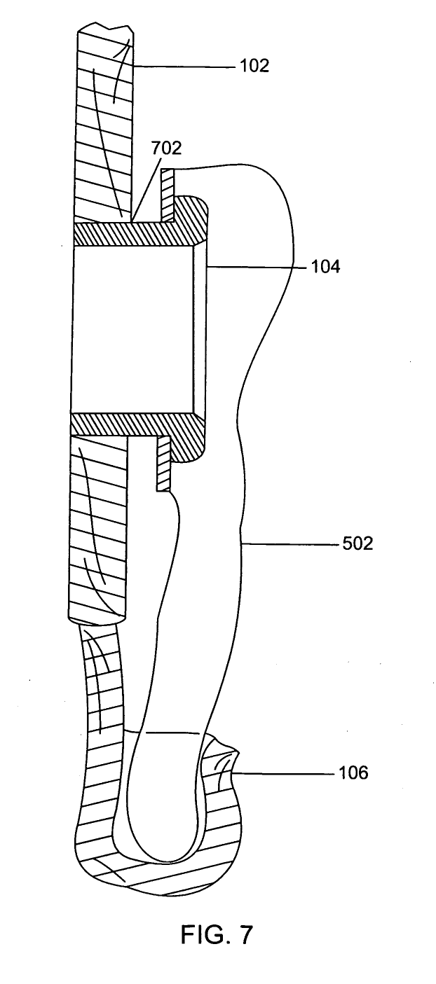

FIG. 7 shows a cross-sectional view of the belly binder illustrating the reinforced opening 104, the support member 106, and the ostomy bag 502. As described previously in FIGS. 1 and 3, the opening 104 is doubly reinforced around its circumference to keep the integrity of the elasticity of the flexible band 102. FIG. 7 illustrates a reinforcement 702 achieved through a pliable plastic material. However, it should be noted that any suitable material can replace the pliable plastic to achieve reinforcement of the opening 104. The reinforcement 702 also provides support to the abdominal area of the wearer, alleviating possible rupture of the ostomy bag 502. Further, the opening 104 gives room to the ostomy bag 502 to fill with excrements, without the pressure of ostomy bag 502 bursting.

As can be seen in FIG. 7, the support member 106 is attached to the lower portion of the flexible band 102. The support member 106 forms a pocket like structure at the bottom of the flexible band 102, to hold the ostomy bag 502 in position while the bag 502 gets filled up with the bodily wastes. In an embodiment, the support member 106 may be formed of the same material that is used for the flexible band 102 (as mentioned in FIG. 1). Examples of such material include, but are not limited to, polyester, spandex, lastol, LYCRA® and the like. Alternatively, the support member 106 may also be formed of cotton or any elastic material known in the art. In some aspects, the material is treated by methods known in the art to provide waterproofing, antimicrobial, deodorant and other desirable characteristics. Preferably, the material is washable. In some aspects, the support member is removable (e.g., replaceable) and/or adjustable (e.g., location or size). It may be formed of a disposable or single-use type medical grade material.

In another embodiment, the support member 106 may comprise a padding to provide a better support to the ostomy bag 502. The padding may be in the form of a foam material that is inserted inside the support member 106 for a better hold and to avoid sagging/contraction of the support member 106.

However, it is not necessary that the support member 106 is attached to the lower portion of the flexible band 102. In a further embodiment, the support member 106 may be attached to the reinforced opening 104. Further, the ostomy bag 502 may be replaced by any medical prosthetic device to provide support to the abdomen. Examples of such prosthetic device include, but are not limited to, a penrose type drainage tube that is used after certain surgical drainage procedures. A penrose drain is a surgical device placed in a wound to drain fluid. It consists of a soft rubber tube placed in a wound area, to prevent the buildup of fluid.

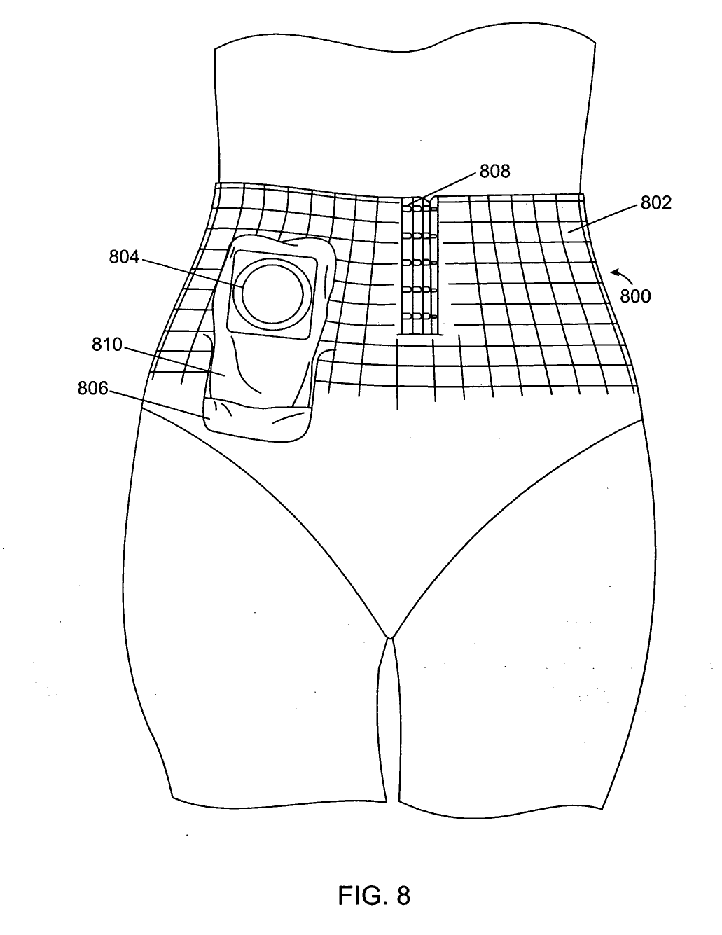

FIG. 8 shows an embodiment of the belly binder 100 in the form of a girdle 800. The girdle 800 comprises a main body 802, an opening 804, a support member 806, a fastener arrangement 808, and an ostomy bag 810. The main body 802 of the girdle 800 is in the shape of a panty and is adapted to confine the hips and abdomen of a wearer. The main body 802 is preferably made of an elastic material, so as to provide good support and fit to a user. Examples of the material used to form the main body 802 include, but are not limited to, polyester, spandex, lastol, LYCRA®, ELASPAN (Invista), CREORA (Hyosung), ROICA (Asahi Kasei), and DORLASTAN (Asahi Kasei), LINEL (Fillattice), and ESPA (Toyobo), and the like. In some aspects, the material is treated by methods known in the art to provide waterproofing, antimicrobial, deodorant and other desirable characteristics. Preferably, the material is washable. In some aspects, the material is disposable or single-use type medical grade material.

The main body. 802 comprises the opening 804. The opening 804 is adapted to receive a medical prosthetic device. In an embodiment, the medical prosthetic device is the ostomy bag 810. The opening 804 can be of any shape. The shape of the opening 804 may be circular, square, rectangular, elliptical etc. The opening 804 is reinforced at its circumference to provide support to the stoma and to maintain integrity of the main body 802. In an embodiment, the opening 804 is reinforced by using a pliable plastic that is placed between two layers of elastic binding material. The opening 804 is of a size such that it can easily receive the ostomy bag 810, without interfering or rupturing the ostomy bag 810. In an embodiment, the opening 804 is such that the diameter of the opening 804 is greater than the diameter of the ostomy bag 810 (attached to the body). The provision of the opening 804 on the surface of the main body 802 makes the ostomy bag 810 accessible, without the removal of the girdle 800.

Further, the main body 802 comprises the support member 806. The support member 806 is such that it forms a pocket that hangs vertically down from the main body 802. The support member 806 holds the ostomy bag 810 in position when the bag 810 fills up from the bodily wastes. Further, the support member 806 protects the stoma from the gravitational pull of the ostomy bag 810. This reduces the risk of bag rupture and/or leakage. In an embodiment, the support member 806 is attached to the reinforced opening 804. Alternatively, the support member 806 may also be attached to the main body 802 of the girdle 800.

Referring to FIG. 8, the girdle 800 further comprises the fastener arrangement 808. The fasteners forming the fastener arrangement 808 are placed along at least a portion of the vertical length of the main body 802. The fasteners securely hold the girdle 800 together and provide a good fit to the wearer. Further, the fasteners prevent a relative movement between the skin of a wearer and the girdle 800. A series of fasteners are arranged along the vertical length of the girdle 800. Examples of the fastener arrangement 808 include but are not limited to, hook and eye arrangement, zipper arrangement, lace-thread arrangement, VELCRO®, and a button-hole arrangement.

FIG. 8 represents a hook and eye arrangement. In an embodiment of the present invention, there is provided three series of eyeholes at one end of the main body 802. The three-series eyeholes are placed at three different locations along the vertical length of the main body 802. This is done in order to provide three different fitting settings to the user for a close and snug fit of the girdle 800. Simultaneously a single series of hooks are placed at the other edge of the main body 802. Although only a single row of hooks is depicted in the exemplary drawings, it is to be appreciated that any number or arrangement of fasteners may be suitable adapted to provide the closing function. Multiple rows of fasteners may increase the compressive forces of the garment or distribute the pulling forces over a greater area to enhance the longevity of the fasteners and decrease wear and tear.

The girdle 800 may be worn by a patient after a full recovery from surgeries like ileostomy, colostomy or urostomy. This embodiment of the girdle 800 provides an opportunity to the patient to wear clothing without the bulkiness of any belly binder.

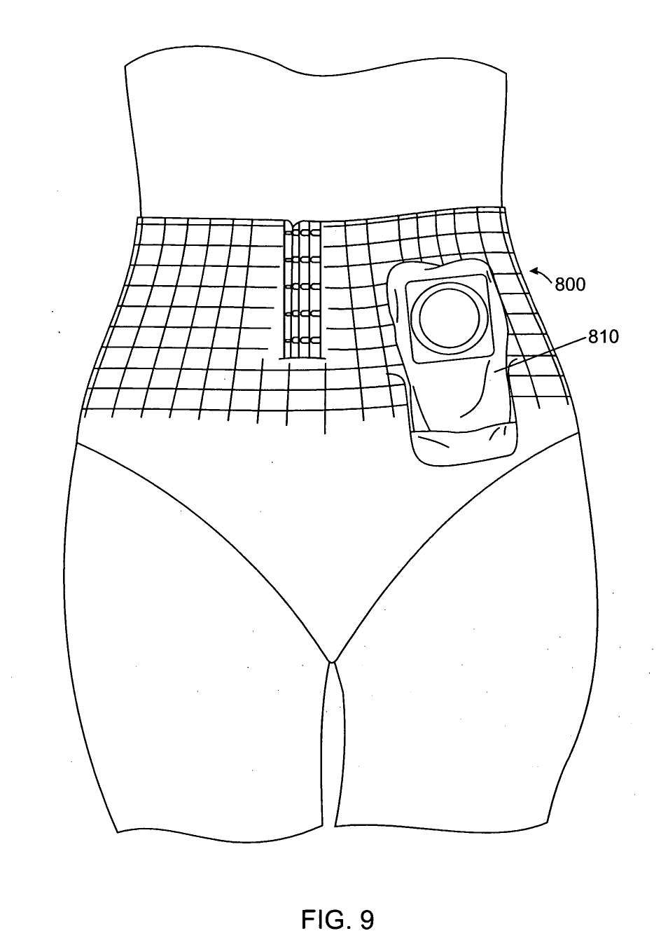

FIG. 9 illustrates another embodiment of the girdle 800. In the shown embodiment, the ostomy bag 810 is placed on the left side of the body of the wearer.

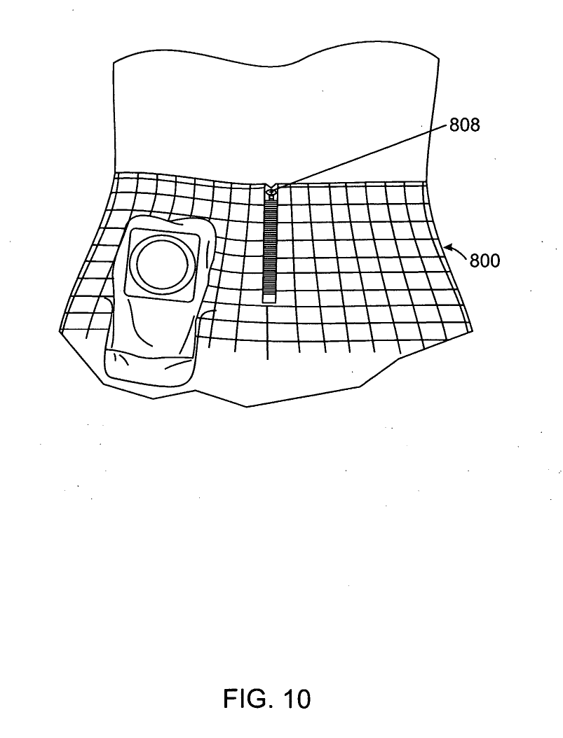

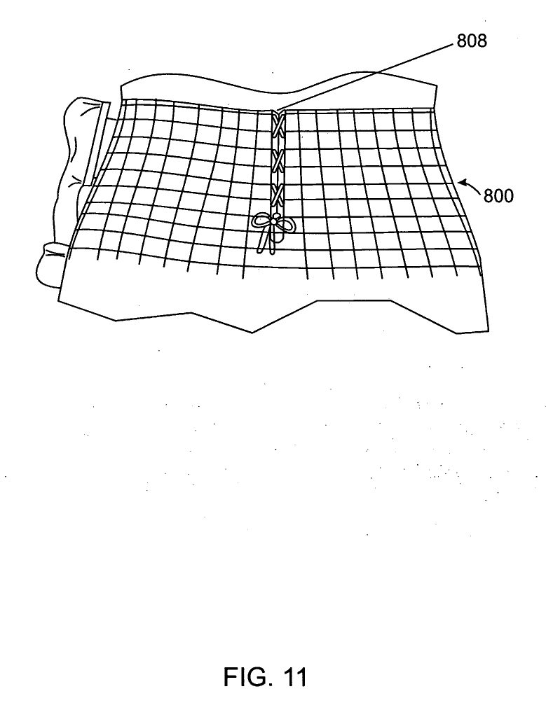

FIGS. 10 and 11 illustrate yet another embodiment of the girdle 800, wherein the fastener arrangement 808 is a zipper arrangement and a lace-thread arrangement respectively.

Numerous alterations of the processes and the methods herein disclosed will suggest themselves to those skilled in the art. However, it is to be understood that the present disclosure relates to the preferred embodiment of the invention which is for purposes of illustration only and not to be construed as a limitation of the invention.

I claim:

1. A belly binder for providing support to an ostomy bag the belly binder comprising:
a. a flexible band having a length, wherein the flexible band is wrapped around a body region requiring support;
b. at least one fastener, wherein the fastener is attached to ends of the flexible band to hold the flexible band in position;
c. an opening in the flexible band, the opening adapted to receive the ostomy bag is disposed about an abdomen; and
d. a support member attached to the flexible band, wherein the support member forms a pocket to hold the ostomy bag.

2. The belly binder of claim 1 further comprising a plurality of fasteners along a width of the flexible band.

3. The belly binder of claim 2, wherein the plurality of fasteners is located at least two different locations along the length of the flexible band to provide different fittings.

4. The belly binder of claim 1, wherein the at least one fastener is a hook and eye arrangement.

5. The belly binder of claim 1, wherein the opening is further adapted to receive a medical prosthetic device.

6. The belly binder of claim 1, wherein the opening is reinforced around the circumference to provide support.

7. The belly binder of claim 1, wherein a diameter of the opening is larger than a diameter of the ostomy bag.

8. The belly binder of claim 1, wherein the flexible band is wrapped around the abdomen.

9. The belly binder of claim 1, wherein the flexible band comprises a heavy duty elastic band.

10. A girdle for providing support to an ostomy bag, the girdle has a length and comprises:
a. a main body wherein the main body is adapted to confine the hips and abdomen of a wearer;
b. at least one fastener, wherein the fastener is connected along a vertical length of the main body, and wherein the fastener holds the girdle in position;
c. an opening in the main body, wherein the opening is adapted to receive the ostomy bag; and
d. a support member attached to the main body, wherein the support member forms a pocket to hold the ostomy bag.

11. The girdle of claim 10, wherein the girdle is a panty girdle.

12. The girdle of claim 10 further comprising a plurality of fasteners attached along at least a portion of the vertical length of the main body.

13. The girdle of claim 12, wherein the plurality of fasteners is located at least two different locations along the length of the girdle to provide different fittings.

14. The girdle of claim 12, wherein the plurality of fasteners is a hook and eye arrangement.

15. The girdle of claim 10, wherein the at least one fastener is a zipper arrangement.

16. The girdle of claim 10, wherein the at least one fastener is a lace arrangement.

17. The girdle of claim 10, wherein the girdle is made of a heavy duty elastic material.

18. The girdle of claim 10, wherein a diameter of the opening is larger than a diameter of the ostomy bag.

19. The girdle of claim 10, wherein the opening is reinforced around the circumference to provide support.
